(19) United States
(12) Patent Application Publication
(10) Pub. No.: US 2025/0381169 A1
(43) Pub. Date: Dec. 18, 2025

(54) METHODS FOR TREATING RENAL DISEASE

(71) Applicant: Arizona Board of Regents on Behalf of the University of Arizona, Tucson, AZ (US)

(72) Inventor: Rick SCHNELLMANN, Tucson, AZ (US)

(21) Appl. No.: 18/877,704

(22) PCT Filed: Jun. 21, 2023
(86) PCT No.: PCT/US2023/068811

§ 371 Date: Dec. 20, 2024

Related U.S. Application Data
(60) Provisional application No. 63/366,704, filed on Jun. 21, 2022.

(51) Int. Cl. A61K 31/404; A61P 13/12

(52) U.S. Cl. CPC A61K 31/404; A61P 13/12

(57) ABSTRACT

Disclosed herein is a method for treating renal disease in a subject that involves treating the subject with an effective amount of a compound of the formula (I). The disclosed method can in some embodiments, be used to treat any renal disease involving mitochondrial dysfunction. For example, in some embodiments, the renal disease involves acute kidney injury. As another example, in some embodiments, the renal disease involves diabetic kidney disease. In some embodiments, the renal disease is a glomerular disease (e.g. focal segmental glomerular sclerosis). In some embodiments, the renal disease is a renal vascular disease.

### CROSS-REFERENCE TO RELATED APPLICATIONS

This application claims benefit of U.S. Provisional Application No. 63/366,704, filed Jun. 21, 2022, which is hereby incorporated herein by reference in its entirety.

### BACKGROUND

Acute kidney injury (AKI) involves rapid renal decline and this occurs in 8-16% of hospitalized patients. AKI can be induced by drugs, sepsis, trauma, and ischemia reperfusion (I/R). AKI causes mitochondrial dysfunction and renal tubular and microvasculature injury but no cure is available. Thus, therapeutics to promote renal recovery are needed.

### SUMMARY

Disclosed herein is a method for treating renal disease in a subject that involves treating the subject with an effective amount of a compound of formula (I):

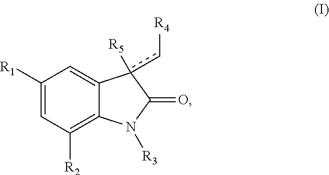

wherein:
R₁ and R₂ are, independently of each other, hydrogen, lower alkyl or halogen;
R₃ is lower alkyl, branched or unbranched, optionally substituted with —CF₃ or piperidine;
R₄ is: (i) phenyl, optionally mono-, bi- or tri-substituted independently with alkoxy, hydroxy, —OC(O)CH₃, —OC(O)CH₂OCH₃, —OC(O)-lower alkyl, —OC(O)NHCH₂CH₂OCH₂CH₂OH, —OSO₂N(CH₃)₂ or —OC(O)N(CH₃)₂;
   (ii) methyl-1H-indazolyl,
   (iii) benzo[d][1,3]dioxolyl,
   (iv) benzo[d] imidazolyl,
   (v) benzoyl-1H-indolyl,
   (vi) benzo[d] oxazolyl,
   (vii) oxazolo[4,5-b]pyridinyl or
   (viii) a 6-membered heteroaryl group having one or more ring carbons replaced by N;
R₅ is hydrogen, hydroxyl, —CH₂-pyridazinyl, —OR₆, —NHR₆ or absent;
R₆ is —C(O)-pyridinyl, —P(O)(OCH₂CH₃)₂, —C(O)CH₂OCH₃, —C(O)N(CH₃)₂, —C(O)—O-1,3-dioxolan-4-yl)methyl, —SO₂-phenylmethyl or —C(O)-phenyl; and
the symbol [symbol] indicates a single or double bond,
or a pharmaceutically acceptable salt thereof.

Therefore, in some embodiments, the compound disclosed herein comprises 5-chloro-1-ethyl-3-(2-hydroxy-3-methoxybenzyl)-2-oxoindolin-3-yl dimethylcarbamate having the formula II (also referred to herein as “MC16”):

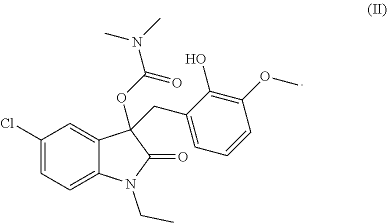 [SMILES: CCN1C(=O)C(Cc2cccc(OC)c2O)(OC(=O)N(C)C)c2cc(Cl)ccc21]

The disclosed method can in some embodiments, be used to treat any renal disease involving mitochondrial dysfunction. For example, in some embodiments, the renal disease involves acute kidney injury. As another example, in some embodiments, the renal disease involves diabetic kidney disease. In some embodiments, the renal disease is a glomerular disease (e.g. focal segmental glomerular sclerosis). In some embodiments, the renal disease is a renal vascular disease.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1D show MC16 stimulates mitochondrial biogenesis. Mitochondrial number increases in kidney cortex of naïve mice after MC16 treatment in vivo. Mice were treated with vehicle, 0.3 mg/kg or 1 mg/kg of MC16 with 2 doses IP (Oh, 24h) and harvested at 48 h.

FIGS. 2A to 2G show MC16 increases mitochondrial and antioxidant proteins. MC16 induces mitochondrial biogenesis pathway proteins and/or associated mRNA. Proteins were measured using immunoblot analysis or PCR to measure mRNA. Bars with different superscripts are significantly different from one another (P<0.05); n=6. FIG. 2G is a table of antioxidant and electron transport chain proteins and associated mRNAs.

FIGS. 3A and 3B show in a mouse AKI model MC16 (cX) promotes recovery of renal function and tubular injury. Impaired glomerular function leads to increase serum creatinine levels. Significant kidney injury leads to increased Kidney Injury Molecule 1 (Kim1) expression (proximal tubular biomarker). Bars with different superscripts are significantly different from one another (P<0.05); n=6.

FIG. 4 illustrates mitochondrial fission/fusion & biogenesis. Fusion involves MFN1, MFN2; fission involves DRP1, pDRP1, and biogenesis involves PGC1a. Damaged mitochondria undergo mitophagy (degradation)

FIGS. 5A and 5B show in a mouse AKI model MC16 (cX) increases MFN1 (fusion). MFN2 did not change during injury or MC16 treatment. Bars with different superscripts are significantly different from one another (P<0.05); n=6.

FIGS. 6A and 6B show in a mouse AKI model that DRP1 and pDRP1 are elevated at 24 h of injury. MC16 (cX) does not have an affect on DRP1 but decreases pDRP1 (fission) at 144 h. Bars with different superscripts are significantly different from one another (P<0.05); n=6.

FIGS. 7A and 7B show in a mouse AKI model MC16 (cX) increases nuclear PGC-1α (biogenesis) but not in the cytosolic fraction. Translocation of PGC-1 into the nucleus is required activity. Bars with different superscripts are significantly different from one another (P<0.05); n=6.

FIGS. 8A to 8F show in a db/db model of diabetic kidney disease MC16 (cX) restores electron transport chain proteins. Bars with different superscripts are significantly different from one another (P<0.05); n=6.

FIGS. 9A and 9B show in a db/db model of diabetic kidney disease MC16 (cX) restores of PGC1α and ATP. Bars with different superscripts are significantly different from one another (P<0.05); n=6.

FIGS. 10A and 10B in a db/db model of diabetic kidney disease show MFN1 (fusion) s is decreased in DB/DB in the presence and absence of MC16 (cX). MFN2 (fusion) was increased in the presence of MC16. Bars with different superscripts are significantly different from one another (P<0.05); n=6.

FIGS. 11A and 11B show MC16 (cX) has no effect on mitochondrial fission proteins. Bars with different superscripts are significantly different from one another (P<0.05); n=6.

FIGS. 12A to 12F show MC16 (cX) increases mitophagy proteins. Bars with different superscripts are significantly different from one another (P<0.05); n=6.

FIG. 13 shows MC16 (cX) does not change glomerular function over time. The mice have yet to show signs of DKD in the timeframe. Bars with different superscripts are significantly different from one another (P<0.05); n=6.

FIG. 14 shows weight loss with MC16 (cX). Bars with different superscripts are significantly different from one another (P<0.05); n=6.

FIG. 15 shows MC16 (cX) improves vascular tight junction proteins. Bars with different superscripts are significantly different from one another (P<0.05); n=6.

FIGS. 16A to 16F show MC16 (cX) does not increase/decrease blood glucose or urine output compared to diabetic mice getting vehicle.

FIGS. 17A to 17E show MC16 (cX) increases body weight similar to that of diabetic mice getting vehicle. MC16 (cX) normalized kidney weight to control mice.

FIGS. 18A to 18C show MC16 (cX) normalizes serum creatinine and KIM1. No effects on NGAL.

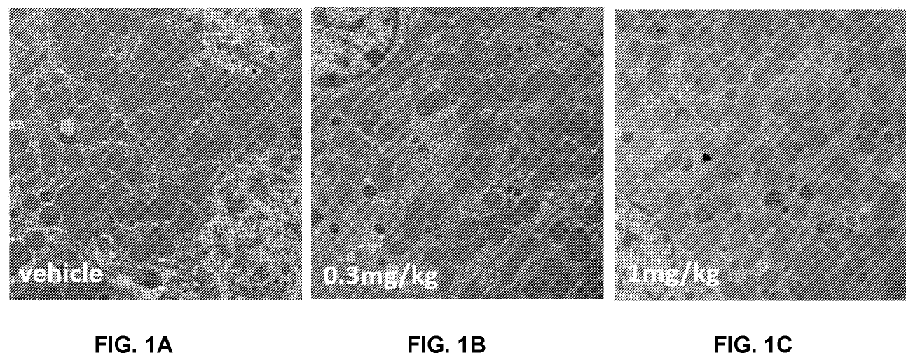

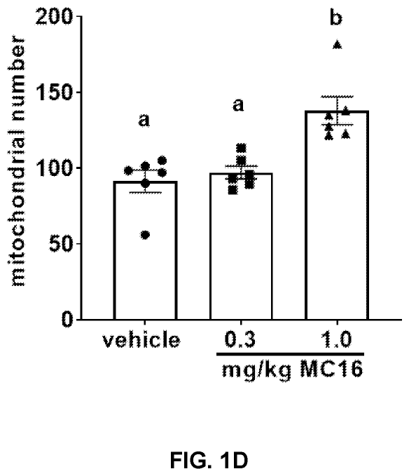

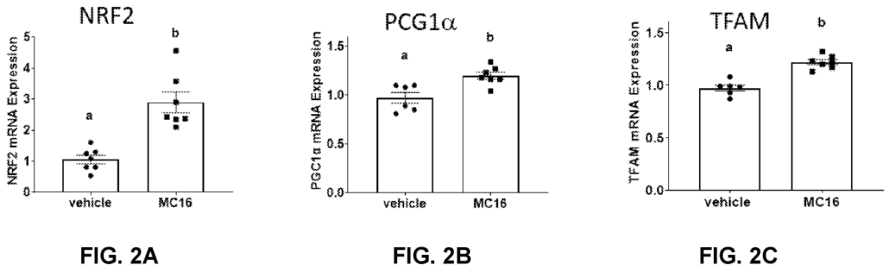

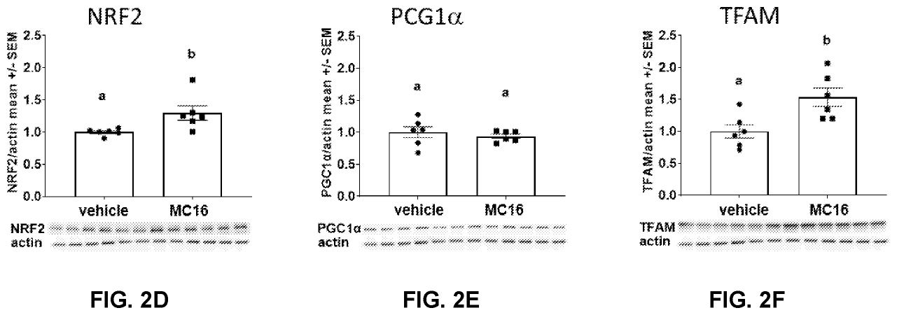

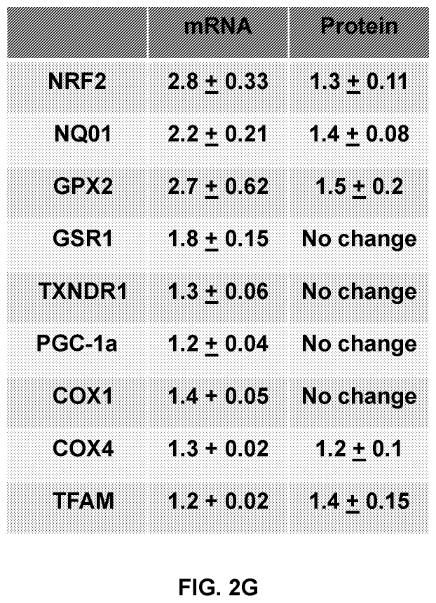

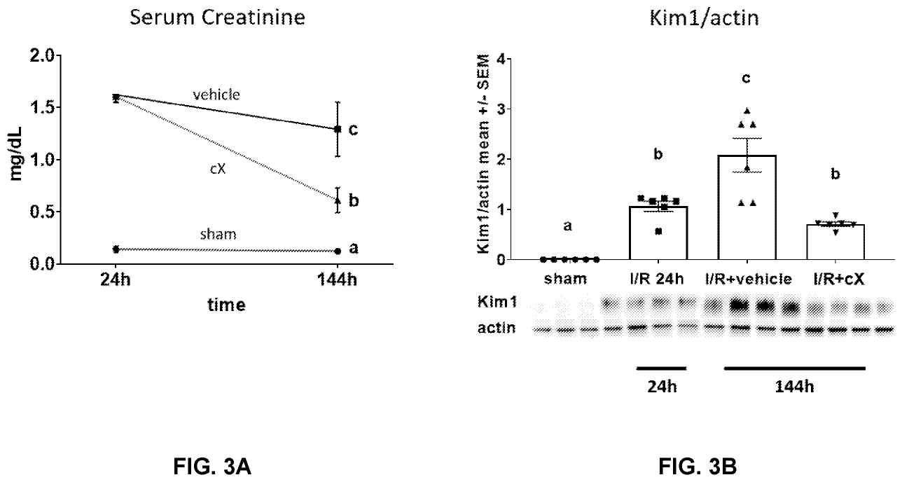

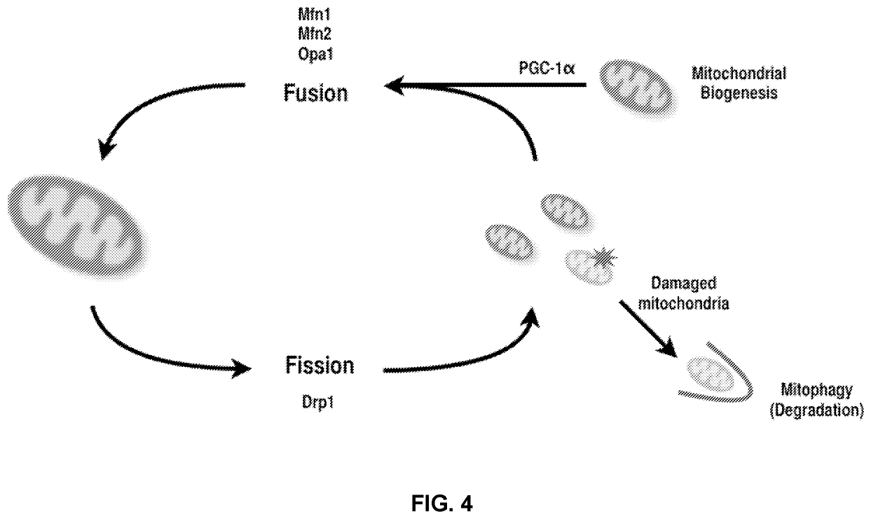

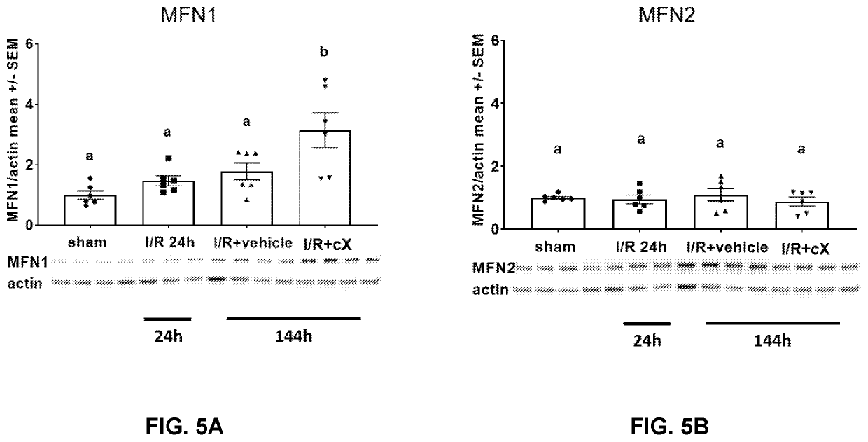

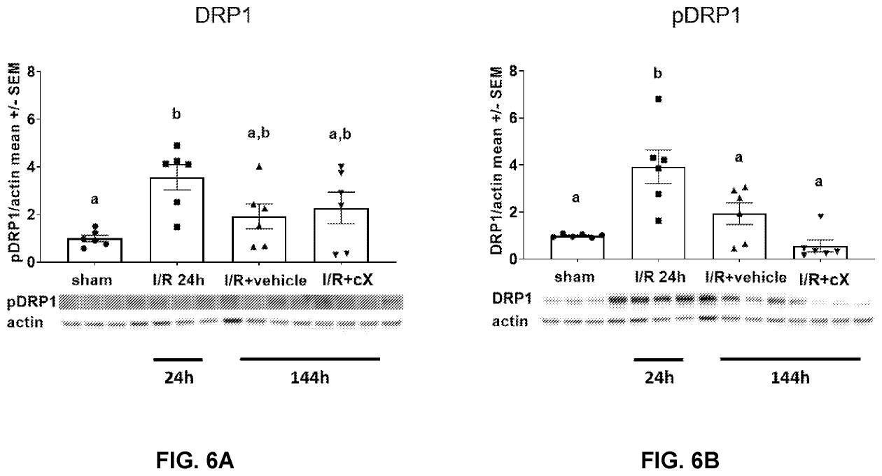

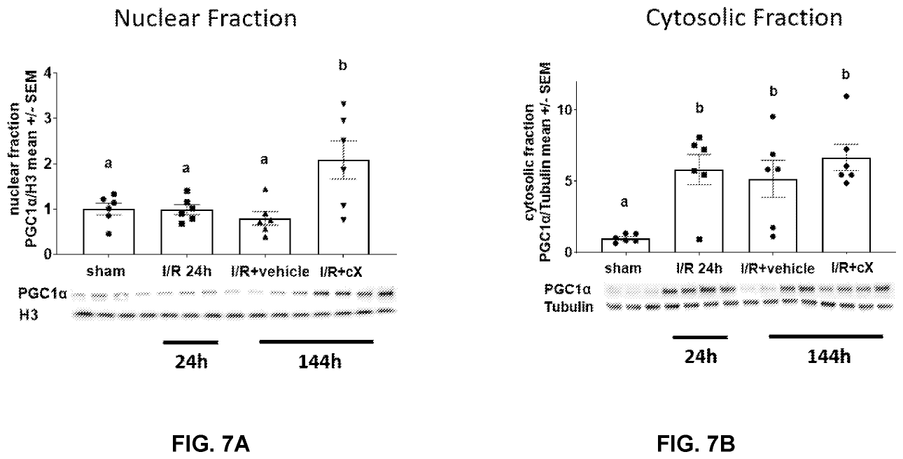

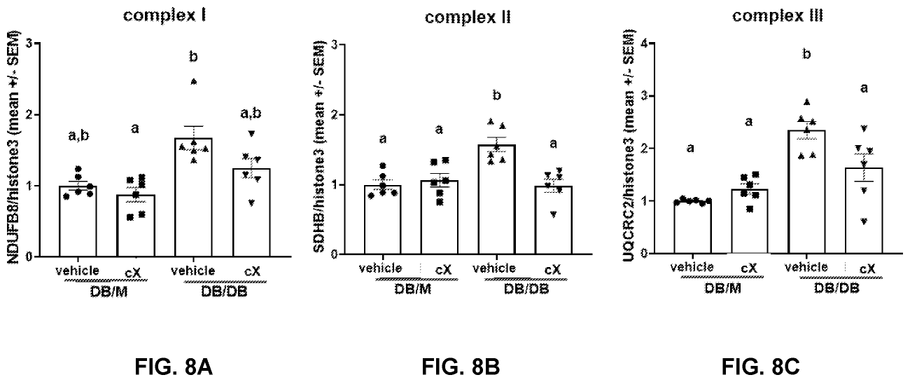

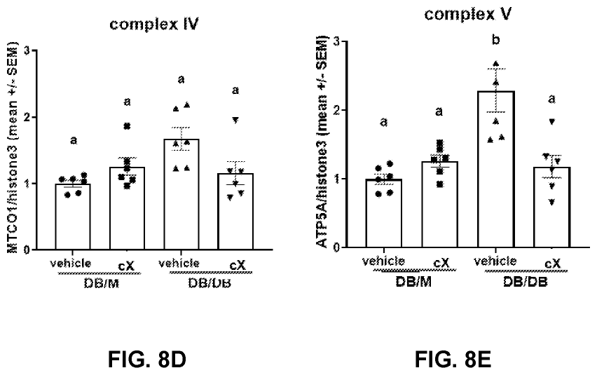

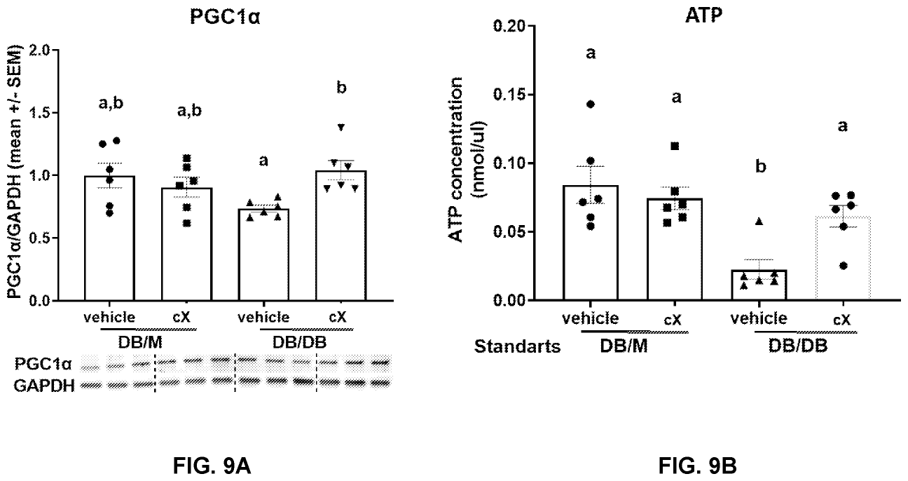

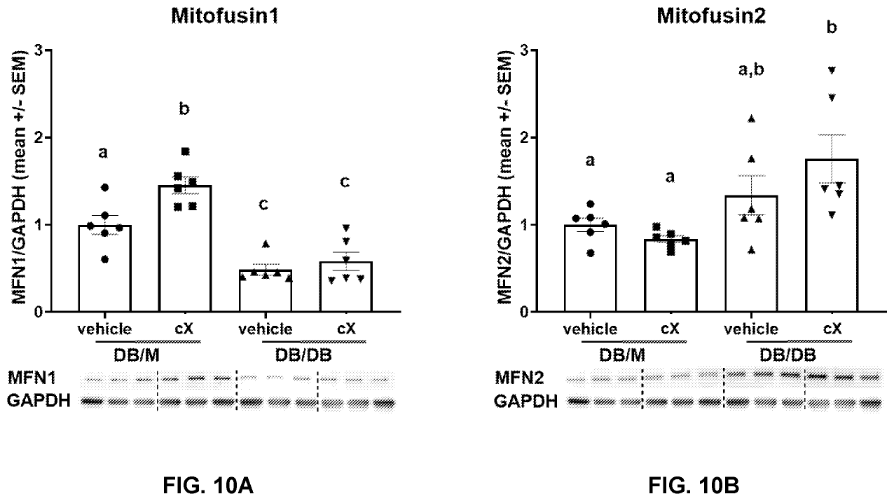

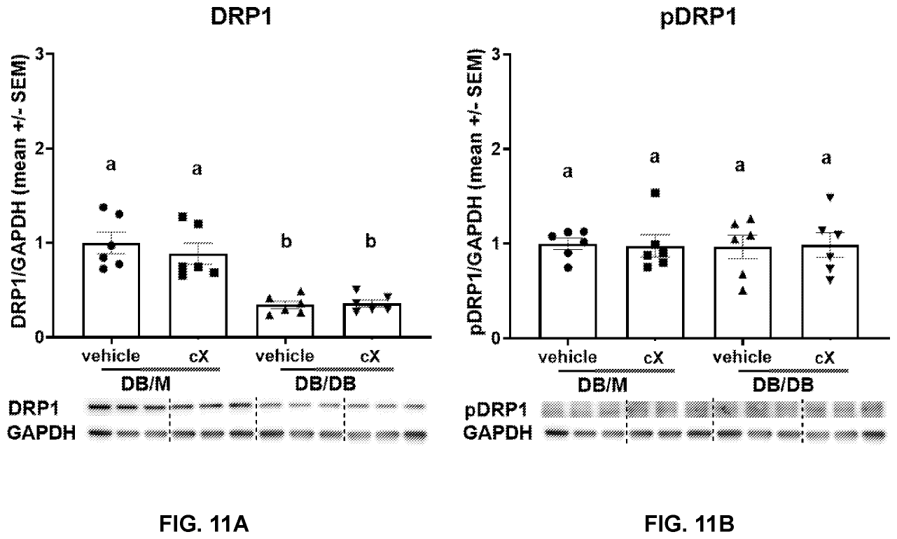

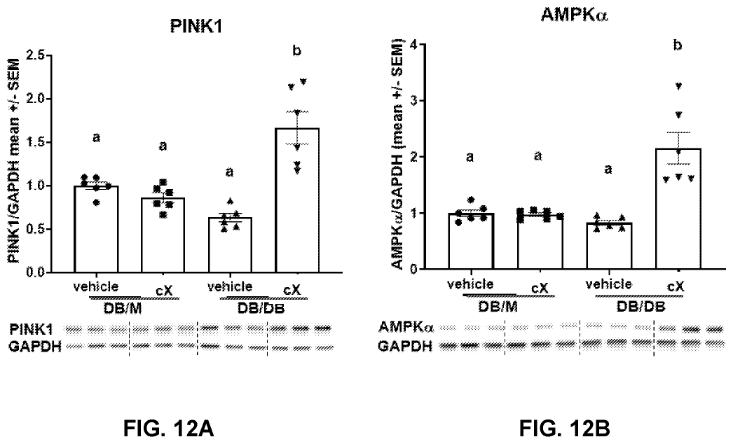

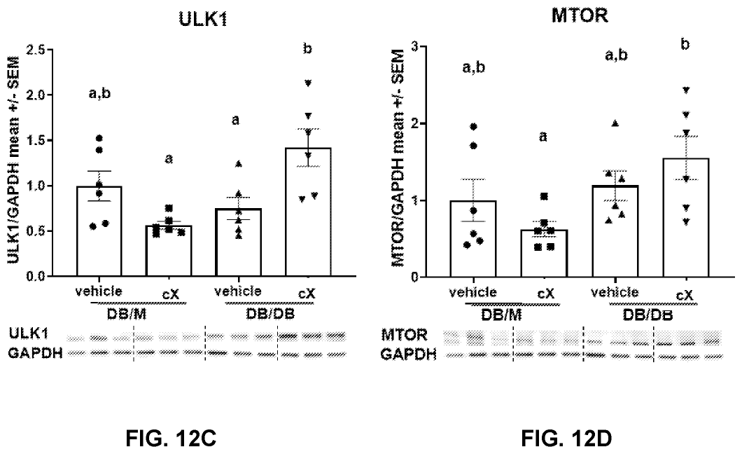

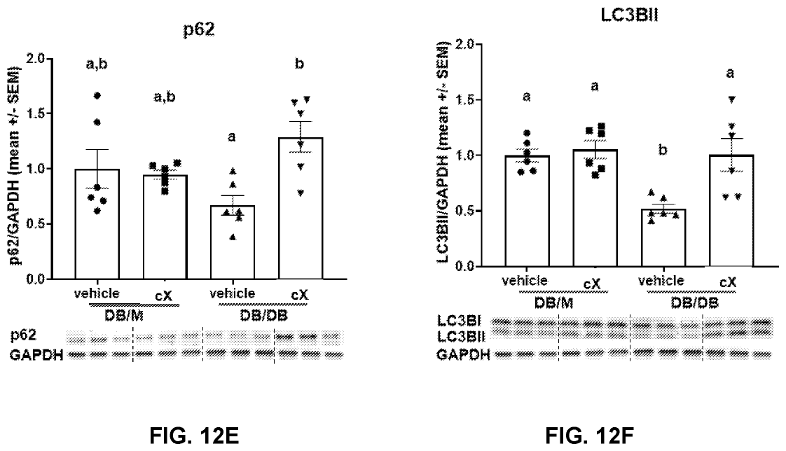

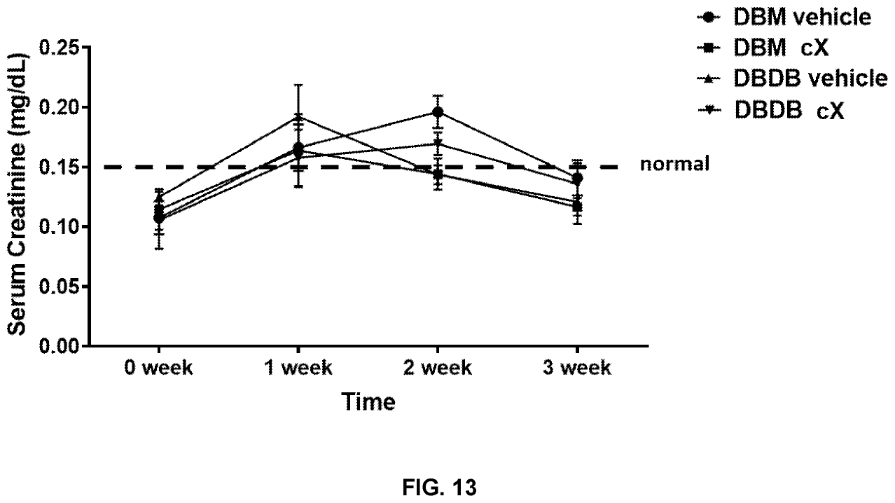

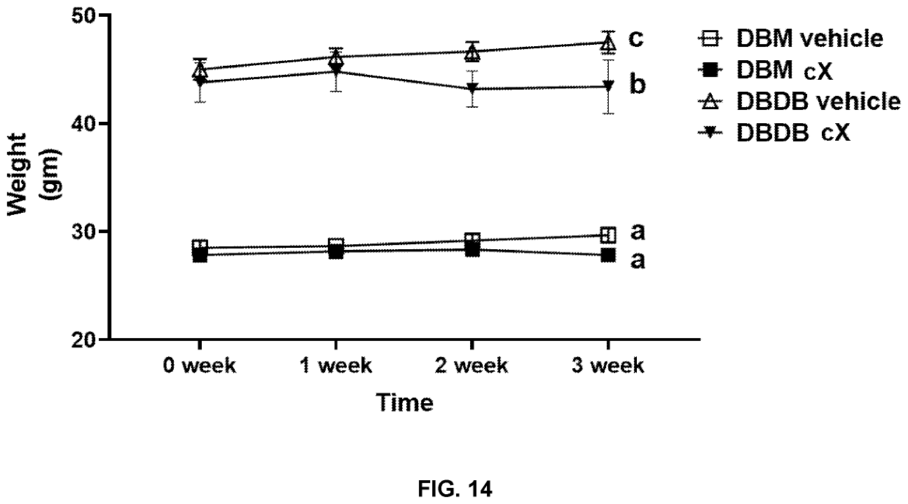

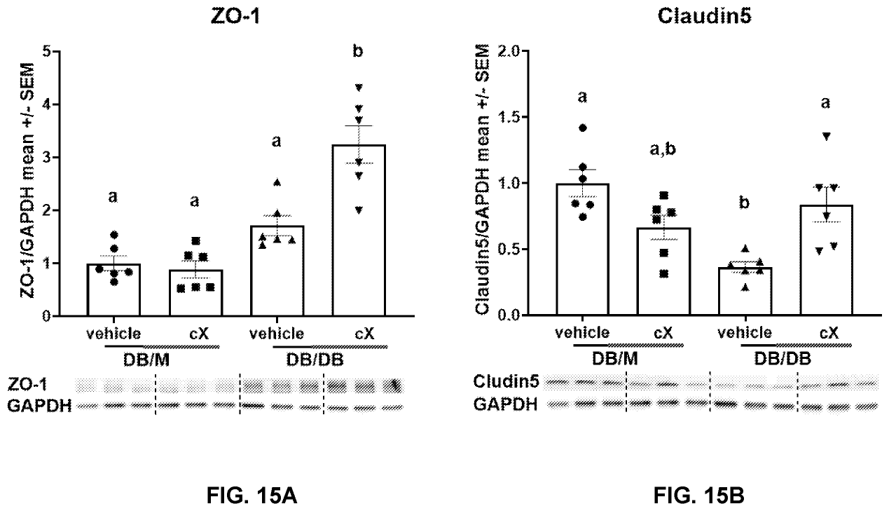

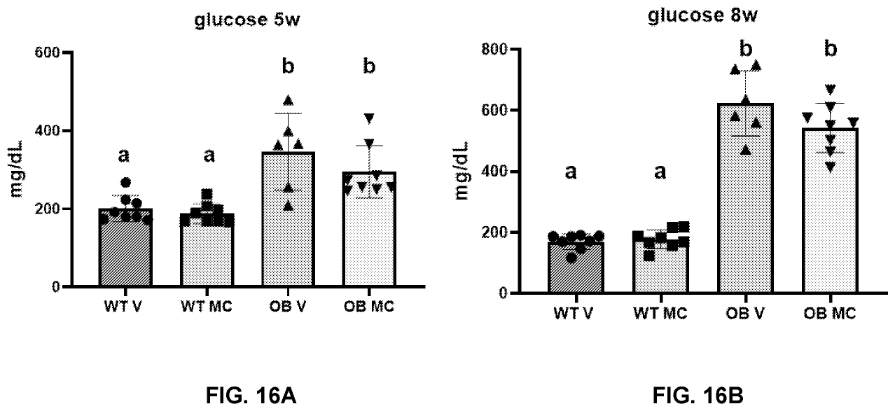

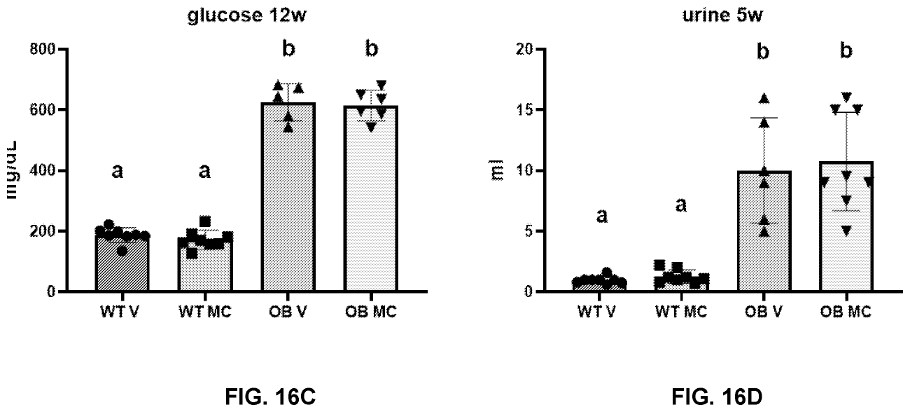

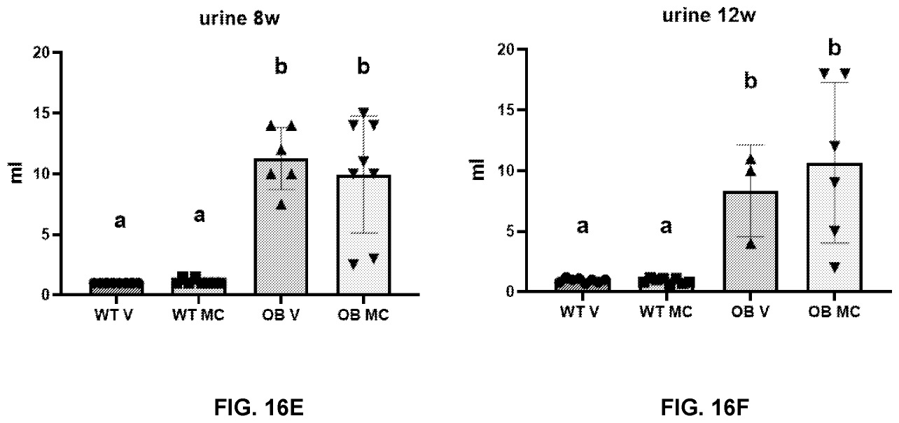

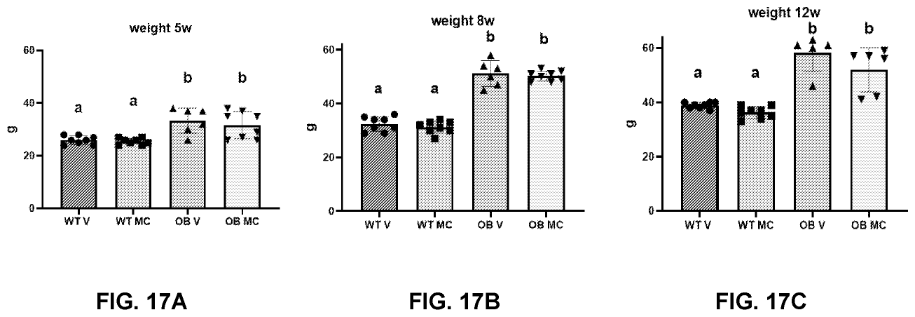

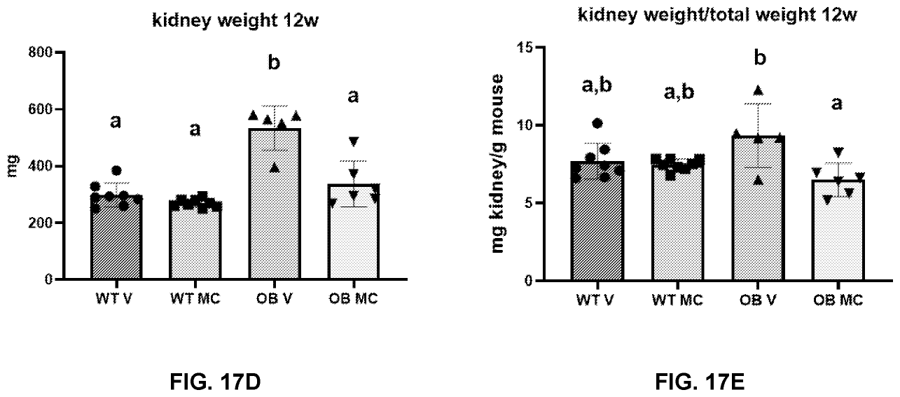

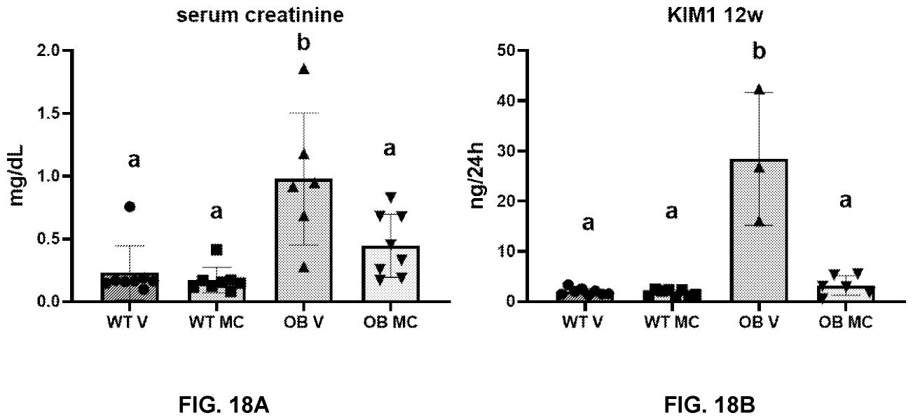

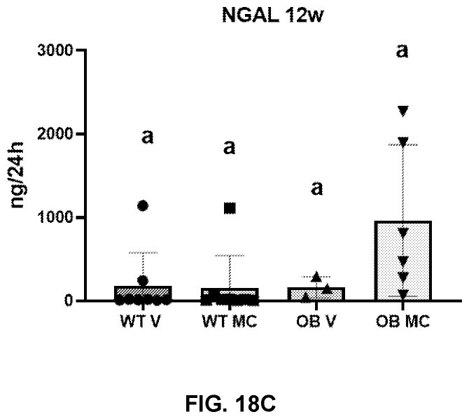

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

As used herein, the term “alkyl”, alone or in combination with other groups, refers to a branched or straight-chain monovalent saturated aliphatic hydrocarbon radical of one to twenty carbon atoms, in one embodiment one to sixteen carbon atoms, in another embodiment one to ten carbon atoms.

As used herein, the term “alkenyl”, alone or in combination with other groups, refers to a straight-chain or branched hydrocarbon residue having an olefinic bond.

The term “cycloalkyl” refers to a monovalent mono- or polycarbocyclic radical of three to ten, in one embodiment three to six, carbon atoms. This term is further exemplified by radicals such as cyclopropyl, cyclobutyl, cyclopentyl, cyclohexyl, cycloheptyl, norbornyl, adamantyl, indanyl and the like. In one embodiment, the “cycloalkyl” moieties can optionally be substituted with one, two, three or four substituents. Each substituent can independently be, alkyl, alkoxy, halogen, amino, hydroxyl or oxygen unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate.

The term “lower alkyl”, alone or in combination with other groups, refers to a branched or straight-chain alkyl radical of one to nine carbon atoms, in another embodiment one to six carbon atoms, in a further embodiment one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthalene, 1,2-dihydronaphthalene, indanyl, 1H-indenyl and the like.

The alkyl, lower alkyl and aryl groups may be substituted or unsubstituted. When substituted, there will generally be, for example, 1 to 4 substituents present. These substituents may optionally form a ring with the alkyl, lower alkyl or aryl group with which they are connected. Substituents may include, for example: carbon-containing groups such as alkyl, aryl, arylalkyl (e.g., substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl (hydroxyl) alkyl), ethers (e.g. alkoxy, aryloxy, alkoxyalkyl, aryloxyalkyl, in another embodiment, for example, methoxy and ethoxy), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, aryl sulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group.

The heteroaryl group described above may be substituted independently with one, two, or three substituents. Substituents may include, for example: carbon-containing groups such as alkyl, aryl, arylalkyl (e.g., substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl (hydroxyl) alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkyl sulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl, benzothiazoyl and carbolinyl).

As used herein, the term “alkoxy” means alkyl-O—; and “alkoyl” means alkyl-CO—. Alkoxy substituent groups or alkoxy-containing substituent groups may be substituted by, for example, one or more alkyl groups.

As used herein, the term “halogen” means a fluorine, chlorine, bromine or iodine radical, in another embodiment a fluorine, chlorine or bromine radical, and in a further embodiment a bromine or chlorine radical.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Representative embodiments include fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminum salts.

As used herein, “diabetic patient” encompasses both Type 1 and Type 2 diabetic patients and “diabetes” encompasses both Type 1 and Type 2 diabetes.

As used herein, “limiting the progression of renal disease” means to reduce or prevent decreases in renal function in those patients receiving treatment relative to diabetic patients not receiving the treatment. Such treatment thus reduces the need for kidney dialysis or transplantation in diabetic patients.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

### Compounds

Disclosed herein is a method for treating renal disease in a subject that involves treating the subject with an effective amount of a compound described in U.S. Pat. No. 10,370,328, which is incorporated by reference for these compounds and methods of making same. For example, in some embodiments compound disclosed herein is a compound of formula (I):

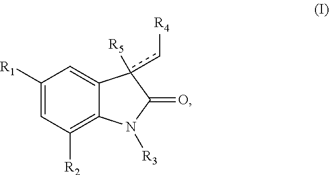

wherein:
R₁ and R₂ are, independently of each other, hydrogen, lower alkyl or halogen;
R₃ is lower alkyl, branched or unbranched, optionally substituted with-CF₃ or piperidine;
R₄ is: (i) phenyl, optionally mono-, bi- or tri-substituted independently with alkoxy, hydroxy, —OC(O)CH₃, —OC(O)CH₂OCH₃, —OC(O)-lower alkyl, —OC(O)NHCH₂CH₂OCH₂CH₂OH, —OSO₂N(CH₃)₂ or —OC(O)N(CH₃)₂;
   (ii) methyl-1H-indazolyl,
   (iii) benzo[d][1,3]dioxolyl,
   (iv) benzo[d]imidazolyl,
   (v) benzoyl-1H-indolyl,
   (vi) benzo[d] oxazolyl,
   (vii) oxazolo[4,5-b]pyridinyl or
   (viii) a 6-membered heteroaryl group having one or more ring carbons replaced by N;
R₅ is hydrogen, hydroxyl, —CH₂-pyridazinyl, —OR₆, —NHR₆ or absent;
R₆ is —C(O)-pyridinyl, —P(O)(OCH₂CH₃)₂, —C(O)CH₂OCH₃, —C(O)N(CH₃)₂, —C(O)—O-1,3-dioxolan-4-yl)methyl, —SO₂-phenylmethyl or —C(O)-phenyl; and
the symbol [symbol] indicates a single or double bond, or a pharmaceutically acceptable salt thereof.

In some embodiments the compound is a compound of formula I wherein R₁ is hydrogen.

In some embodiments the compound is a compound of formula I wherein R₁ is lower alkyl or halogen.

In some embodiments the compound is a compound of formula I wherein R₁ is methyl or chlorine.

In some embodiments the compound is a compound of formula I wherein R₂ is hydrogen.

In some embodiments the compound is a compound of formula I wherein R₃ is unsubstituted lower alkyl.

In some embodiments the compound is a compound of formula I wherein R₃ is methyl, ethyl, pentyl, butyl, isobutyl, isopentyl or methylpentyl.

In some embodiments the compound is a compound of formula I wherein R₃ is trifluoroethyl.

In some embodiments the compound is a compound of formula I wherein R₄ is unsubstituted phenyl.

In some embodiments the compound is a compound of formula I wherein R₄ is phenyl mono-, bi or trisubstituted independently with alkoxy or hydroxy.

In some embodiments the compound is a compound of formula I wherein R₄ is phenyl bisubstituted independently with alkoxy, hydroxy, —OC(O)CH₃, —OC(O)CH₂OCH₃, —OC(O)-lower alkyl, —OC(O)NHCH₂CH₂OCH₂CH₂OH, —OSO₂N(CH₃)₂ or —OC(O)N(CH₃)₂.

In some embodiments the compound is a compound of formula I wherein R₄ is methyl-1H-indazolyl, benzo[d][1,3]dioxolyl, benzo[d]imidazolyl, benzoyl-1H-indolyl, benzo[d]oxazolyl or oxazolo[4,5-b]pyridinyl.

In some embodiments the compound is a compound of formula I wherein R₄ is a 6-membered heteroaryl group having one or more ring carbons replaced by N.

In some embodiments the compound is a compound of formula I wherein R₄ is pyrimidinyl, pyrazinyl, pyridazinyl or pyridinyl.

In some embodiments the compound is a compound of formula I wherein R₅ is hydrogen or hydroxy.

In some embodiments the compound is a compound of formula I wherein R₅ is —OR₆ or —NHR₆.

In some embodiments the compound is a compound of formula I wherein R₆ is —C(O)-pyridinyl or —C(O)-phenyl.

In some embodiments the compound is a compound of formula I wherein R₆ is —C(O)CH₂OCH₃, —C(O)N(CH₃)₂ or —C(O)—O-1,3-dioxolan-4-yl)methyl.

In some embodiments the compound is a compound of formula I wherein R⁶ is —P(o)(OCH₂CH₃)₂.

In some embodiments the compound is: 3-hydroxy-5-methyl-1-(2,2,2-trifluoroethyl)-3-(3,4,5-trimethoxybenzyl) indolin-2-one; 2-((3-hydroxy-5-methyl-1-(4-methylpentyl)-2-oxoindolin-3-yl)methyl)-5-methoxyphenyl isonicotinate; 2-((3-hydroxy-5-methyl-1-(4-methylpentyl)-2-oxoindolin-3-yl)methyl)-5-methoxyphenyl acetate; 2-((3-hydroxy-5-methyl-1-(4-methylpentyl)-2-oxoindolin-3-yl)methyl)-5-methoxyphenyl 2-methoxyacetate; 5-chloro-1-ethyl-3-(2-hydroxy-3-methoxybenzyl)-2-oxoindolin-3-yl isonicotinate; 5-chloro-1-ethyl-3-(2-hydroxy-3-methoxybenzyl)-2-oxoindolin-3-yl picolinate; 5-chloro-1-ethyl-3-(2-hydroxy-3-methoxybenzyl)-2-oxoindolin-3-yl diethyl phosphate; 2-((5-chloro-1-ethyl-3-hydroxy-2-oxoindolin-3-yl)methyl)-6-methoxyphenyl 2-methoxyacetate; 2-((5-chloro-1-ethyl-3-hydroxy-2-oxoindolin-3-yl)methyl)-6-methoxyphenyl butyrate; 5-chloro-1-ethyl-3-(2-hydroxy-3-methoxybenzyl)-2-oxoindolin-3-yl dimethylcarbamate; (1,3-dioxolan-4-yl) methyl (5-chloro-1-ethyl-3-(2-hydroxy-3-methoxybenzyl)-2-oxoindolin-3-yl) carbonate; 5-chloro-1-ethyl-3-(2-(((2-(2-hydroxyethoxy)ethyl) carbamoyl)oxy)-3-methoxybenzyl)-2-oxoindolin-3-yl benzoate; 2-((5-chloro-1-ethyl-2-oxoindolin-3-yl)methyl)-6-methoxyphenyl dimethylcarbamate; 3-hydroxy-1,5-dimethyl-3-((1-methyl-1H-indazol-4-yl)methyl) indolin-2-one; 1-butyl-3-((1-methyl-1H-indazol-4-yl)methyl)-2-oxoindolin-3-yl benzoate; 3-(benzo[d][1,3]dioxol-4-ylmethyl)-5-chloro-2-oxo-1-propylindolin-3-yl benzoate; 3-(benzo[d][1,3]dioxol-4-ylmethyl)-1-ethyl-3-hydroxy-5-methylindolin-2-one; 3-(benzo[d][1,3]dioxol-4-ylmethyl)-1-butyl-3-hydroxyindolin-2-one; 3-(benzo[d][1,3]dioxol-4-ylmethyl)-3-hydroxy-1-isobutylindolin-2-one; 3-(benzo[d][1,3]dioxol-4-ylmethyl)-1-isopentyl-2-oxoindolin-3-yl benzoate; 3-(benzo[d][1,3]dioxol-4-ylmethyl)-1-isobutyl-5-methyl-2-oxoindolin-3-yl benzoate; 3-(benzo[d][1,3]dioxol-4-ylmethyl)-1-isopentyl-5-methyl-2-oxoindolin-3-yl benzoate; 3-((1H-benzo[d]imidazol-4-yl)methyl)-5-chloro-1-ethyl-3-hydroxyindolin-2-one; 1-ethyl-3-(2-hydroxy-3,4-dimethoxybenzyl)-5-methyl-2-oxoindolin-3-yl dimethylcarbamate; 3-((1-benzoyl-1H-indol-3-yl)methyl)-2-oxo-1-propylindolin-3-yl dimethylcarbamate; 3-((1H-benzo[d]imidazol-4-yl)methyl)-5-chloro-1-methyl-2-oxoindolin-3-yl dimethylcarbamate; 5-chloro-1-ethyl-3-(pyrimidin-4-ylmethyl) indolin-2-one; 5-methyl-1-ethyl-3-(pyrimidin-4-ylmethylene) indolin-2-one; 5-chloro-1-ethyl-3-hydroxy-3-(pyrazin-2-ylmethyl) indolin-2-one; 1-ethyl-5-methyl-3-(pyrazin-2-ylmethylene) indolin-2-one; 1-propyl-3-(pyridazin-3-ylmethylene) indolin-2-one; 1-propyl-3-(pyridazin-3-ylmethyl) indolin-2-one; 1,5-dimethyl-3-(pyridazin-3-ylmethyl) indolin-2-one; N-(5-chloro-2-oxo-1-propyl-3-(pyridazin-3-ylmethyl) indolin-3-yl) nicotinamide; 1-methyl-3-(pyridazin-3-ylmethylene) indolin-2-one; 3-((1H-benzo[d]imidazol-2-yl)methylene)-5-chloro-1-ethylindolin-2-one; 5-chloro-3-hydroxy-1-propyl-3-(pyrazin-2-ylmethyl) indolin-2-one; 5-chloro-3-hydroxy-1-methyl-3-(pyridin-4-ylmethyl) indolin-2-one; 5-chloro-3-hydroxy-1-propyl-3-(pyridazin-4-ylmethyl) indolin-2-one; 3-hydroxy-1-methyl-3-(pyridin-4-ylmethyl) indolin-2-one; (E)-3-((1H-benzo[d]imidazol-2-yl)methylene)-5-chloro-1-methylindolin-2-one; N-(3-((1H-benzo[d]imidazol-2-yl)methyl)-1-methyl-2-oxoindolin-3-yl) nicotinamide; 1-ethyl-3-hydroxy-3-(pyridazin-3-ylmethyl) indolin-2-one; 1-ethyl-5-methyl-3,3-bis (pyridazin-4-ylmethyl) indolin-2-one; 1-propyl-3,3-bis (pyridazin-4-ylmethyl) indolin-2-one; 1-ethyl-3-hydroxy-5-methyl-3-(pyridazin-3-ylmethyl) indolin-2-one; N-(3-((1H-benzo[d]imidazol-2-yl)methyl)-5-chloro-1-ethyl-2-oxoindolin-3-yl)isonicotinamide; 3-(benzo[d]oxazol-2-ylmethylene)-5-chloro-1-methylindolin-2-one; N-(3-((1H-benzo[d]imidazol-2-yl)methyl)-5-chloro-2-oxo-1-propylindolin-3-yl)benzamide; 1-methyl-3-(oxazolo[4,5-b]pyridin-2-ylmethylene) indolin-2-one; 5-chloro-2-oxo-1-(2-(piperidin-1-yl)ethyl)-3-(pyridin-2-ylmethyl) indolin-3-yl dimethylcarbamate; N-(3-((3-methoxypyridin-2-yl)methyl)-2-oxo-1-propylindolin-3-yl)benzamide; N-(5-chloro-1-ethyl-2-oxo-3-(pyridin-2-ylmethyl) indolin-3-yl)-4-methylbenzenesulfonamide; 3-(1-ethyl-5-methyl-3-((1-methyl-1H-indazol-4-yl)methyl)-2-oxoindolin-3-yl)-1,1-dimethylurea; 1, 1-dimethyl-3-(3-((1-methyl-1H-indazol-4-yl)methyl)-2-oxo-1-propylindolin-3-yl) urea; 3-(3-(benzo[d][1,3]dioxol-4-ylmethyl)-2-oxo-1-propylindolin-3-yl)-1,1-dimethylurea; 3-(3-(benzo[d][1,3]dioxol-4-ylmethyl)-5-chloro-1-methyl-2-oxoindolin-3-yl)-1,1-dimethylurea; 3-(3-(benzo[d][1,3]dioxol-4-ylmethyl)-5-chloro-2-oxo-1-propylindolin-3-yl)-1, 1-dimethylurea; 3-(1,5-dimethyl-2-oxo-3-(3,4,5-trimethoxybenzyl) indolin-3-yl)-1,1-dimethylurea; 3-(1-ethyl-2-oxo-3-(3,4,5-trimethoxybenzyl) indolin-3-yl)-1,1-dimethylurea; 3-(3-((1-benzoyl-1H-indol-3-yl)methyl)-1-ethyl-2-oxoindolin-3-yl)-1,1-dimethylurea; 3-(3-((1-benzoyl-1H-indol-3-yl)methyl)-1-methyl-2-oxoindolin-3-yl)-1,1-dimethylurea; 3-(3-((1-benzoyl-1H-indol-3-yl)methyl)-2-oxo-1-propylindolin-3-yl)-1,1-dimethylurea; 3-(3-((1-benzoyl-1H-indol-3-yl)methyl)-5-methyl-2-oxo-1-propylindolin-3-yl)-1,1-dimethylurea; or 2-((5-chloro-1-ethyl-3-hydroxy-2-oxoindolin-3-yl)methyl)-6-methoxyphenyl dimethylsulfamate.

In some embodiments, the compound is 5-chloro-1-ethyl-3-(2-hydroxy-3-methoxybenzyl)-2-oxoindolin-3-yl dimethylcarbamate, having formula (II) (also referred to herein as “MC16”).

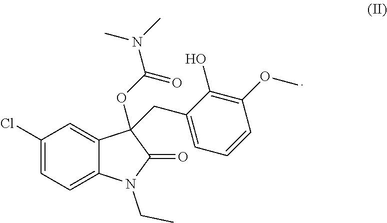 [SMILES: CCN1C(=O)C(Cc2cccc(OC)c2O)(OC(=O)N(C)C)c2cc(Cl)ccc21]

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered, for example, ocularly, orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions.

The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are representative liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. In one embodiment, the therapeutically effective amount is in an amount of from about 1 mg to about 500 mg per day.

It will be appreciated, that the compounds disclosed herein may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds disclosed herein can be prepared beginning with commercially available starting materials and utilizing general synthetic techniques and procedures known to those skilled in the art. Chemicals may be purchased from companies such as for example Aldrich, Argonaut Technologies, VWR and Lancaster. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram I Mettler Toledo Instrument Newark, Del. Biotage, ISCO and Analogix columns are pre-packed silica gel columns used in standard chromatography.

An effective amount of any one of the compounds disclosed herein or a combination of any of the compounds or a pharmaceutically acceptable salt thereof, can be administered via any of the usual and acceptable methods known in the art, either singly or in combination. The compounds or compositions can thus be administered, for example, ocularly, orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions.

The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are representative liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. In one embodiment, the therapeutically effective amount is in an amount of from about 1 mg to about 500 mg per day.

It will be appreciated, that the compounds of general formula I may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds disclosed herein can be prepared beginning with commercially available starting materials and utilizing general synthetic techniques and procedures known to those skilled in the art. Chemicals may be purchased from companies such as for example Aldrich, Argonaut Technologies, VWR and Lancaster. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

### Methods of Treatment

Disclosed herein is a method for treating renal disease in a subject that involves treating the subject with an effective amount of a compound disclosed herein. The disclosed methods can in some embodiments treat or reduce the progression rate, frequency, and/or severity of a kidney disease and kidney-disease-related disease events, particularly treating, preventing, or reducing the progression rate, frequency, and/or severity of one or more complications of a kidney disease.

In general, treatment or prevention of a disease or condition as described in the present disclosure is achieved by administering a compound disclosed herein in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

The kidneys maintain many features of the blood, including volume, PH balance, electrolyte concentrations, and blood pressure, as well as bearing responsibility for toxin and waste filtration. These functions depend upon the intricate structure of the kidney nephrons, constant flow of blood through the various capillaries of the kidney, and the regulation of the kidney by signals from the rest of the body, including endocrine hormones. Problems with kidney function manifest by direct mechanisms (e.g., genetic defects, infection, or toxin exposure) and by indirect mechanisms progressively proceeding from long term stressors like hypertrophy and hyperfiltration (themselves often a result of more direct insults to kidney function). Due to the central role of the kidney in blood maintenance and waste secretion, kidney-associated disease manifestations are many and varied; they can be reviewed in Harrison's Principles of Internal Medicine, 18th edition, McGraw Hill, N.Y., Part 13, Chp 277-289.

In some embodiments, the disclosed methods can be applied to various kidney-associated diseases or conditions. As used herein, “kidney-associated disease or condition” can refer to any disease, disorder, or condition that affects the kidneys or the renal system. Examples of kidney-associated diseases or conditions include, but are not limited to, chronic kidney diseases (or failure), acute kidney diseases (or failure), primary kidney diseases, non-diabetic kidney diseases, glomerulonephritis, interstitial nephritis, diabetic kidney diseases, diabetic chronic kidney disease, diabetic nephropathy, glomerulosclerosis, rapid progressive glomerulonephritis, renal fibrosis, Alport syndrome, IDDM nephritis, mesangial proliferative glomerulonephritis, membranoproliferative glomerulonephritis, crescentic glomerulonephritis, renal interstitial fibrosis, focal segmental glomerulosclerosis, membranous nephropathy, minimal change disease, pauci-immune rapid progressive glomerulonephritis, IgA nephropathy, polycystic kidney disease, Dent's disease, nephrocytinosis, Heymann nephritis, polycystic kidney disease (e.g., autosomal dominant (adult) polycystic kidney disease and autosomal recessive (childhood) polycystic kidney disease), acute kidney injury, nephrotic syndrome, renal ischemia, podocyte diseases or disorders, proteinuria, glomerular diseases, membranous glomerulonephritis, focal segmental glomerulonephritis, pre-eclampsia, eclampsia, kidney lesions, collagen vascular diseases, benign orthostatic (postural) proteinuria, IgM nephropathy, membranous nephropathy, sarcoidosis, diabetes mellitus, kidney damage due to drugs, Fabry's disease, aminoaciduria, Fanconi syndrome, hypertensive nephrosclerosis, interstitial nephritis, acute interstitial nephritis, Sickle cell disease, hemoglobinuria, myoglobinuria, Wegener's Granulomatosis, Glycogen Storage Disease Type 1, chronic kidney disease, chronic renal failure, low Glomerular Filtration Rate (GFR), nephroangiosclerosis, lupus nephritis, A CA-positive pauci-immune crescentic glomerulonephritis, chronic allograft nephropathy, nephrotoxicity, renal toxicity, kidney necrosis, kidney damage, glomerular and tubular injury, kidney dysfunction, nephritic syndrome, acute renal failure, chronic renal failure, proximal tubal dysfunction, acute kidney transplant rejection, chronic kidney transplant rejection, non-IgA mesangioproliferative glomerulonephritis, postinfectious glomerulonephritis, vasculitides with renal involvement of any kind, any hereditary renal disease, any interstitial nephritis, renal transplant failure, kidney cancer, kidney disease associated with other conditions (e.g., hypertension, diabetes, and autoimmune disease), Deal's disease, iiepliio ylinosis, Heymann nephritis, a primary kidney disease, a collapsing glomerulopathy, a dense deposit disease, a cryoglobulinemia-associated glomerulonephritis, an Henoch-Schonlein disease, a postinfectious glomerulonephritis, a bacterial endocarditis, a microscopic polyangitis, a Churg-Strauss syndrome, an anti-GBM-antibody mediated glomerulonephritis, amyloidosis, a monoclonal immunoglobulin deposition disease, a fibrillary glomerulonephritis, an immunotactoid glomerulopathy, ischemic tubular injury, a medication-induced tubulo-interstitial nephritis, a toxic tubulo-interstitial nephritis, an infectious tubulointerstitial nephritis, a bacterial pyelonephritis, a viral infectious tubulo-interstitial nephritis which results from a polyomavirus infection or an HIV infection, a metabolic-induced tubulo-interstitial disease, a mixed connective disease, a cast nephropathy, a crystal nephropathy which may results from urate or oxalate or drug-induced crystal deposition, an acute cellular tubulo-interstitial allograft rejection, a tumoral infiltrative disease which results from a lymphoma or a post-transplant lymphoproliferative disease, an obstructive disease of the kidney, vascular disease, a thrombotic microangiopathy, a nephroangiosclerosis, an atheroembolic disease, a mixed connective tissue disease, a polyarteritis nodosa, a calcineurin-inhibitor induced-vascular disease, an acute cellular vascular allograft rejection, an acute humoral allograft rejection, early renal function decline (ERFD), end stage renal disease (ESRD), renal vein thrombosis, acute tubular necrosis, acute interstitial nephritis, established chronic kidney disease, renal artery stenosis, ischemic nephropathy, uremia, drug and toxin-induced chronic tubulointerstitial nephritis, reflux nephropathy, kidney stones, Goodpasture's syndrome, normocytic normochromic anemia, renal anemia, diabetic chronic kidney disease, IgG4-related disease, von Hippel-Lindau syndrome, tuberous sclerosis, nephronophthisis, medullary cystic kidney disease, renal cell carcinoma, adenocarcinoma, nephroblastoma, lymphoma, leukemia, hyposialylation disorder, chronic cyclosporine nephropathy, renal reperfusion injury, renal dysplasia, azotemia, bilateral arterial occlusion, acute uric acid nephropathy, hypovolemia, acute bilateral obstructive uropathy, hypercalcemic nephropathy, hemolytic uremic syndrome, acute urinary retention, malignant nephrosclerosis, postpartum glomerulosclerosis, scleroderma, non-Goodpasture's anti-GBM disease, microscopic polyarteritis nodosa, allergic granulomatosis, acute radiation nephritis, post-streptococcal glomerulonephritis, Waldenstrom's macroglobulinemia, analgesic nephropathy, arteriovenous fistula, arteriovenous graft, dialysis, ectopic kidney, medullary sponge kidney, renal osteodystrophy, solitary kidney, hydronephrosis, microalbuminuria, uremia, haematuria, hyperlipidemia, liypoalbuminaemia, lipiduria, acidosis, edma, tubulointerstitial renal fibrosis, hypertensive sclerosis, juxtaglomerular cell tumor, Fraser syndrome, Horseshoe kidney, renal tubular dysgenesis, hypokalemia, hypomagnesemia, hypercalcemia, hypophosphatemia, uromodul in-associated kidney disease, Nail-patella syndrome, lithium nephrotoxity, TNF-alpha nephrotoxicity, honeybee resin related renal failure, sugarcane harvesting acute renal failure, complete LCAT deficiency, Fraley syndrome, Page kidney, reflux nephropathy, Bardet-Biedl syndrome, collagenofibrotic glomerulopathy, Dent disease, Denys-Drash syndrome, congenital nephrotic syndrome, immunotactoid glomerulopathy, fibronextin glomerulopathy, Galloway Mowat syndrome, lipoprotein glomerulopathy, MesoAmerican nephropathy, beta-thalassemia renal disease, haemolytic uraemic syndrome, Henoch-Schonlein-Purpura disease, retroperitoneal fibrosis, polyarteritis nodose, cardiorenal syndrome, medullary kidney disease, renal artery stenosis, uromodulin kidney disease, and hyperkalemia.

In some embodiments, a compound disclosed herein may be used to treat or prevent chronic kidney disease, optionally in combination with one or more supportive therapies for treating chronic kidney disease. In some embodiments, a compound disclosed herein may be used to treat or prevent one or more complications (symptoms or manifestations) of chronic kidney disease (e.g., tissue damage, inflammation, and/or fibrosis), optionally in combination with one or more supportive therapies for treating chronic kidney disease. In some embodiments, a compound disclosed herein may be used to treat or prevent end-stage kidney failure, optionally in combination with one or more supportive therapies for treating end-stage kidney disease. Chronic kidney disease (CKD), also known as chronic renal disease, is a progressive loss in renal function over a period of months or years. The symptoms of worsening kidney function may include feeling generally unwell and experiencing a reduced appetite. Often, chronic kidney disease is diagnosed as a result of screening of people known to be at risk of kidney problems, such as those with high blood pressure or diabetes and those with a blood relative with CKD. This disease may also be identified when it leads to one of its recognized complications, such as cardiovascular disease, anemia, or pericarditis. Recent professional guidelines classify the severity of CKD in five stages, with stage 1 being the mildest and usually causing few symptoms and stage 5 being a severe illness with poor life expectancy if untreated. Stage 5 CKD is often called end-stage kidney disease, end-stage renal disease, or end-stage kidney failure, and is largely synonymous with the now outdated terms chronic renal failure or chronic kidney failure; and usually means the patient requires renal replacement therapy, which may involve a form of dialysis, but ideally constitutes a kidney transplant. CKD is initially without specific symptoms and is generally only detected as an increase in serum creatinine or protein in the urine. As the kidney function decreases, various symptoms may manifest as described below. Blood pressure may be increased due to fluid overload and production of vasoactive hormones created by the kidney via the renin-angiotensin system, increasing one's risk of developing hypertension and/or suffering from congestive heart failure. Urea may accumulate, leading to azotemia and ultimately uremia (symptoms ranging from lethargy to pericarditis and encephalopathy). Due to its high systemic circulation, urea is excreted in eccrine sweat at high concentrations and crystallizes on skin as the sweat evaporates (“uremic frost”). Potassium may accumulate in the blood (hyperkalemia with a range of symptoms including malaise and potentially fatal cardiac arrhythmias). Hyperkalemia usually does not develop until the glomerular filtration rate falls to less than 20-25 ml/min/1.73 m², at which point the kidneys have decreased ability to excrete potassium. Hyperkalemia in CKD can be exacerbated by acidemia (which leads to extracellular shift of potassium) and from lack of insulin. Erythropoietin synthesis may be decreased causing anemia. Fluid volume overload symptoms may occur, ranging from mild edema to life-threatening pulmonary edema.

Hyperphosphatemia, due to reduced phosphate excretion, may occur generally following the decrease in glomerular filtration. Hyperphosphatemia is associated with increased cardiovascular risk, being a direct stimulus to vascular calcification. Hypocalcemia may manifest, which is generally caused by stimulation of fibroblast growth factor-23. Osteocytes are responsible for the increased production of FGF23, which is a potent inhibitor of the enzyme 1-alpha-hydroxylase (responsible for the conversion of 25-hydroxycholecalciferol into 1,25-dihydroxyvitamin D3). Later, this progresses to secondary hyperparathyroidism, renal osteodystrophy, and vascular calcification that further impairs cardiac function. Metabolic acidosis (due to accumulation of sulfates, phosphates, uric acid etc.) may occur and cause altered enzyme activity by excess acid acting on enzymes; and also increased excitability of cardiac and neuronal membranes by the promotion of hyperkalemia due to excess acid (acidemia). Acidosis is also due to decreased capacity to generate enough ammonia from the cells of the proximal tubule. Iron deficiency anemia, which increases in prevalence as kidney function decreases, is especially prevalent in those requiring haemodialysis. It is multifactoral in cause, but includes increased inflammation, reduction in erythropoietin, and hyperuricemia leading to bone marrow suppression. People with CKD suffer from accelerated atherosclerosis and are more likely to develop cardiovascular disease than the general population. Patients afflicted with CKD and cardiovascular disease tend to have significantly worse prognoses than those suffering only from the latter.

The progression of renal disease can be measured in various ways, including the following: (a) Proteinuria (ie: increased loss of protein into the urine; often assessed by measurement of albumin levels (ie: “albuminuria”)); (b) Impaired glomerular filtration (ie: kidney function to clear substances from blood; can be measured, for example, by creatinine (ie: “impaired creatinine clearance”), inulin, or urea clearance); (c) Increased levels of serum creatinine; and (d) increased levels of urinary transforming growth factor beta (TGF-β).

In some embodiments, the methods further involve administering one or more supportive therapies to limit the progression of renal disease in a human diabetic patient. Such therapeutics include, but are not limited to, angiotensin converting enzyme inhibitors (ACE-I), angiotensin receptor blockers (ARB), beta-blockers, aldose reductase inhibitors, calcium blockers, diuretics, glycosaminoglycans, incretin mimetics, insulin, insulin sensitizers, statins, fibrates, glucose uptake inhibitors, sulfonylureas, superoxide dismutase (SOD) and SOD mimetics, thiamine pyrophosphate and its prodrugs, transketolase inhibitors, other AGE inhibitors that can mechanistically complement post-Amadori-inhibitors, and protein kinase C inhibitors. The further therapeutic can be administered together as a single formulation with or separately from the compound disclosed herein. The patient supportive therapies can in some embodiments treat or alleviate one or more symptoms, such as high blood pressure (e.g., using angiotensin-converting enzyme (ACE) inhibitors or angiotensin II receptor blockers or a water pill (diuretic), optionally with a low-salt diet), high cholesterol levels (e.g., using statins), anemia (e.g., using hormone erythropoietin, optionally with iron supplement), swelling (e.g., using diuretics), lack of fluids in blood (e.g., with intravenous (IV) fluid supplement), lack of calcium or bone failure (e.g., with calcium and/or vitamin D supplement, or a phosphate binder to lower the blood phosphate level and to protect calcification of blood vessels), high blood potassium level (e.g., using calcium, glucose or sodium polystyrene sulfonate (Kayexalate, Kionex) to lower potassium levels), toxin accumulation (e.g., by hemodialysis and/or peritoneal dialysis), etc. In addition, kidney transplant may be also used as an additional therapy. Some exemplary medications for kidney diseases are Lasix® (furosemide), Demadex® (torsemide), Edecrin® (ethacrynic acid), and sodium edecrin.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: MC16 Promotes Recovery of Renal Function Through Mitochondrial Biogenesis

### Background

Acute kidney injury (AKI) involves rapid renal decline and this occurs in 8-16% of hospitalized patients. AKI can be induced by drugs, sepsis, trauma, and ischemia reperfusion (I/R). AKI causes mitochondrial dysfunction and renal tubular and microvasculature injury but no cure is available. Thus, therapeutics to promote renal recovery are needed.

### Methods

Control and treated mice subjected to renal I/R were treated with MC16 (0.3 mg/kg) or vehicle 24 h after injury and then daily for 120 h (euthanized at 144 h). Using electron microscopy, mitochondria were counted. Vascular permeability was assessed with Evan's blue dye leakage and proteins were measured in renal cortices using immunoblot. Serum creatinine was also measured.

### Results

MC16 induced mitochondria biogenesis 1.4-fold in renal cortices. At 144 h, serum creatinine decreased 41% in I/R group and 72% with I/R+MC16 treatment. Kim1 increased in I/R group and decreased 50% in the I/R+MC16 group. PGC-1α increased in the I/R+MC16 group. Complex I, II, III proteins decreased in I/R group while Complex IV and V recovered completely. Evan's blue dye leakage increased 2.5-fold in I/R group and was restored in the I/R+MC16 group. Tight junction proteins ZO-1 and Claudin5 decreased in I/R group and recovered in the I/R+MC16 group.

### Conclusion

MC16 given after AKI in mice induces mitochondrial biogenesis, decreases vascular and tubular injury, and improves renal function recovery.

### Example 2: MC16 Promotes Recovery of Renal Function Through Mitochondrial Biogenesis and Dynamics and Energetics in the Diabetic Renal Proximal Tubule and Kidney

Diabetes is a prevalent metabolic disease that contributes to approximately 50% of all end-stage renal disease and has limited treatment options. Here, the effects of MC16 on mitochondrial dysfunction and dynamics in renal proximal tubule cells (RPTCs) treated with high glucose and in a mouse model of type 2 diabetes was assessed. RPTCs exposed to 17 mM glucose exhibited increased electron transport chain (ETC) complex I, II, III, and V protein levels and reduced ATP levels and uncoupled oxygen consumption rate compared with RPTCs cultured in the absence of glucose or osmotic controls after 96 h. ETC proteins, ATP, and oxygen consumption rate were restored in RPTCs treated with MC16. RPTCs exposed to high glucose had increased phospho-dynamin-related protein 1 (Drp1), a mitochondrial fission protein, and decreased mitofusin 1 (Mfn1), a mitochondrial fusion protein. MC16 treatment restored phospho-Drp1 and Mfn1 to control levels.

Db/db and nondiabetic (db/m) mice (10 wk old) were treated with MC16 or vehicle for 3 wk and euthanized. Db/db mice showed increased renal cortical ETC protein levels in complexes I, III, and V and decreased ATP; these changes were prevented by MC16. Phospho-Drp1 was increased and Mfn1 was decreased in db/db mice, and MC16 restored both to control levels. Together, these findings demonstrate that hyperglycemic conditions in vivo and exposure of RPTCs to high glucose similarly alter mitochondrial bioenergetic and dynamics profiles and that treatment with MC16 can reverse these effects. MC16 may be a promising strategy for treating early stages of diabetic kidney disease.

### Example 3: Organ Aging: Metabolomics of Drug-Induced Mitochondrial Biogenesis In Aged Mouse Kidneys

### BACKGROUND

Mitochondrial dysfunction is important in the aging process of many organs. We have shown that MC16, a drug candidate, induces MB in mouse renal cortices and improves renal function in response to acute kidney injury. Thus, we hypothesized that stimulation of mitochondrial biogenesis (MB) in aged mouse kidneys would improve global metabolomics in the kidney cortex.

### Methods

Aged 22-month-old male C57B/6J mice were administered 1.0 mg/kg of MC16 or normal saline every other day over a 21-day interval (n=8-9/group). Seven-month-old C57B/6J male mice (controls) were administered normal saline every other day over a 21-day interval (n=7). Kidneys were harvested and snap frozen in liquid nitrogen. Renal cortex biopsies were analyzed by Metabolon for global metabolomic analyses. Briefly, samples were processed and divided into four fractions: two for analysis by RP/UPLC-MS/MS with (+) ion mode electrospray ionization (IMESI), one for RP/UPLC-MS/MS with (−) IMESI, and one for HILIC/UPLC-MS/MS with (−) IMESI. Following mass normalization, log-transformation, and computational imputation, Welch's two-sample t-test was used to identify metabolites that differed significantly between treatment groups. A p-value of p≤0.05 and a false discovery rate of q<0.10 were utilized to identify global metabolite alteration and correct for multiple comparisons, respectively.

### Results

Total, 1,010 biochemicals (BCs) were detected in the global metabolomic dataset. Of these BCs, 441 were statistically different in saline-treated ‘old’ mice compared to saline-treated ‘young’ control mice, with 276 BCs increased and 165 BCs decreased. Additionally, 343 BCs we identified that were statistically different in MC16-treated ‘old’ mice compared to saline-treated ‘young’ control mice, with 214 BCs increased, and 129 BCs decreased. Between these two groups, 140 BCs were statistically different in the MC16-treated ‘old’ mice group compared to the saline-treated ‘old’ mice group, with 72 BCs increased, and 68 BCs decreased. Saline-treated ‘old’ mice displayed reduced 1,5-anhydroglucitol (1,5-AG) and elevated glucose-6-phosphate, fructose-6-phosphate, and dihydroxyacetone phosphate (DHAP) compared to saline-treated ‘young’ control mice, indicative of enhanced glycolysis. Interestingly, MC16-treated ‘old’ mice had no statistical changes in glycolysis metabolites compared to saline-treated ‘young’ control mice. Furthermore, while both groups had elevated carnitine levels, saline-treated ‘old’ mice derived a majority of their carnitine species from leucine, isoleucine, and valine amino acid metabolism, while MC16-treated ‘old’ mice derived a majority of their carnitine species from fatty acid metabolism, indicative of impaired fatty acid metabolism in the saline-treated ‘old’ mice groups. Additionally, saline-treated ‘old’ mice displayed reduced levels of diacylglycerols, monoacylglycerols, as well as acyl glycine containing fatty-acids, and elevated levels of phosphatidylcholine (PC), phosphatidylethanolamine (PE), plasmalogens, and secondary bile acids compared to saline-treated ‘young’ control mice. These metabolic lipid alterations were not observed in MC16-treated ‘old’ mice. Furthermore, MC16-treated ‘old’ mice displayed an increase in long chain polyunsaturated fatty acids (n3 and n6) compared to saline-treated ‘old’ and ‘young’ mice controls.

### Conclusion

These data reveal that global metabolomics can be utilized to identify various age-related kidney metabolic alterations within the renal cortices of mice. Additionally, we identified 140 statistically significant metabolic alterations within the renal cortices between saline-treated ‘old’ mice and MC16-treated ‘old’ mice compared to saline-treated ‘young’ mice controls. Interestingly, in the saline-treated ‘old’ mice we observed a marked increase in the levels of glycolysis metabolites glucose-6-phosphate, fructose-6-phosphate, and DHAP compared to saline-treated ‘young’ mice controls, 1.80-, 2.51-, and 2.04-fold, respectively. Importantly, MC16-treated ‘old’ mice had no statistical changes in glycolysis metabolites compared to saline-treated ‘young’ control mice and displayed a 32%, 24%, and 20% reduction in glycolysis metabolites glucose-6-phosphate, fructose-6-phosphate, and DHAP, respectively, when compared to saline-treated ‘old’ mice. This approach allows identification and monitoring of age-related and disease-related metabolic changes within the renal cortex of mice and examine potential pharmacological agents that may alter/blunt the progression of age-related renal dysfunction in future studies.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating renal disease in a subject, comprising treating the subject with an effective amount of a compound of formula (I): 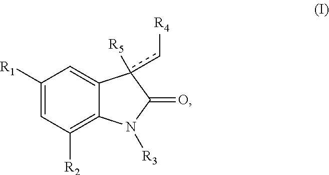
wherein:
R₁ and R₂ are, independently of each other, hydrogen, lower alkyl or halogen;
R₃ is lower alkyl, branched or unbranched, optionally substituted with —CF₃ or piperidine;
R₄ is: (i) phenyl, optionally mono-, bi- or tri-substituted independently with alkoxy, hydroxy, —OC(O)CH₃, —OC(O)CH₂OCH₃, —OC(O)-lower alkyl, —OC(O)NHCH₂CH₂OCH₂CH₂OH, —OSO₂N(CH₃)₂ or —OC(O)N(CH₃)₂;
(ii) methyl-1H-indazolyl,
(iii) benzo[d][1,3]dioxolyl,
(iv) benzo[d]imidazolyl,
(v) benzoyl-1H-indolyl,
(vi) benzo[d]oxazolyl,
(vii) oxazolo[4,5-b]pyridinyl or
(viii) a 6-membered heteroaryl group having one or more ring carbons replaced by N;
R₅ is hydrogen, hydroxyl, —CH₂-pyridazinyl, —OR₆, —NHR₆ or absent;
R₆ is —C(O)-pyridinyl, —P(O)(OCH₂CH₃)₂, —C(O)CH₂OCH₃, —C(O)N(CH₃)₂, —C(O)—O-1,3-dioxolan-4-yl)methyl, —SO₂-phenylmethyl or —C(O)-phenyl; and
the symbol [symbol] indicates a single or double bond,
or a pharmaceutically acceptable salt thereof.

2. The method of claim 1, wherein the compound comprises 5-chloro-1-ethyl-3-(2-hydroxy-3-methoxybenzyl)-2-oxoindolin-3-yl dimethylcarbamate having the formula:

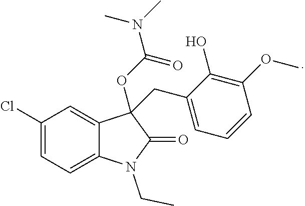 [SMILES: CCN1C(=O)C(Cc2cccc(OC)c2O)(OC(=O)N(C)C)c2cc(Cl)ccc21]

3. The method of claim 1, wherein the renal disease comprises acute kidney injury.

4. The method of claim 1, wherein the renal disease is diabetic kidney disease.

5. The method of claim 1, wherein the renal disease is a glomerular disease.

6. The method of claim 4, wherein the renal disease is focal segmental glomerular sclerosis.

7. The method of claim 1, wherein the renal disease is a renal vascular disease.
